(19) United States
(12) Patent Application Publication
(10) Pub. No.: US 2004/0087480 A1
(43) Pub. Date: May 6, 2004

(54) Calcium channel target for controlling epidermal keratinocyte growth in psoriasis, and basal and squamous cell carcinomas

(75) Inventors: Stanley G. Rane, Lindenhurst, IL (US); Masakazu Kobayashi, Glenview, IL (US); Vlasios Manaves, Westchester, IL (US); Wuxuan Qin, Hangzhou city (CN)

Correspondence Address: OBLON, SPIVAK, MCCLELLAND, MAIER & NEUSTADT, P.C., 1940 DUKE STREET, ALEXANDRIA, VA, 22314, US

(73) Assignee: Fujisawa Pharmaceutical Co. Ltd., Osaka (JP)

(21) Appl. No.: 10/641,158

(22) Filed: Aug. 15, 2003

Related U.S. Application Data
(60) Provisional application No. 60/404,153, filed on Aug. 19, 2002.

(51) Int. Cl. A61K 31/00

(52) U.S. Cl. 514/1

(57) ABSTRACT

Methods and products that modulate the activity of the human epithelial calcium channel type 2 (hECaC2). Modulation of hECaC2 activity permits control of cellular differentiation and proliferation. Such methods and products may be applied to control hyperplastic skin growth in psoriasis and basal and squamous cell carcinomas or to promote tissue repair. Methods for identifying ligands for hECaC2, as well as methods for identifying hECaC2 polypeptides with functional activity.

### CROSS-REFERENCE TO RELATED APPLICATION(S)

This application claims priority to U.S. Provisional Application No. 60/404,153, filed Aug. 19, 2003, the entire contents of which are hereby incorporated by reference.

### BACKGROUND OF THE INVENTION

1. Field of Invention

Methods and products that modulate the activity of the human epithelial calcium channel type 2 (hECaC2). Modulation of hECaC2 activity permits control of cellular differentiation and proliferation. Such methods and products may be applied to control hyperplastic skin growth in psoriasis and basal and squamous cell carcinomas, or to promote tissue repair. Methods for identifying ligands for hECaC2, as well as methods for identifying hECaC2 polypeptides with functional activity are also described.

2. Description of Related Art

Influx of calcium across the keratinocyte plasma membrane is a critical signaling element involved in cellular differentiation in the skin epidermis (Dotto, 1999 Crit Rev Oral Biol Med 10:442-457), yet the gateway for this calcium signal has not been identified at the molecular level. Identifying this calcium entry pathway, and thus a critical control point for keratinocyte growth, will aid in developing treatments for skin diseases that are characterized by epidermal hyperplasia, a condition in which skin keratinocytes both proliferate too rapidly and differentiate poorly. Such diseases include psoriasis, and basal and squamous cell carcinomas. Psoriasis, estimated to affect up to 7 million Americans, afflicts sufferers with mild to extreme discomfort, enhanced susceptibility to secondary infections, and psychological impact due to disfigurement of the affected areas (Lebwohl and Ali, 2001 J Am Acad Dermatol 45:487-498). Basal cell carcinomas (BCC) and squamous cell carcinomas (SCC) of the skin represent at least one-third of all cancers diagnosed in the US each year. More than 1 million new cases are reported annually and incidence is increasing. Despite being relatively non-aggressive, slow-growing cancers, BCCs are capable of significant local tissue destruction and disfigurement. SCCs are more aggressive and thus present even greater complications. Further, given that 80% of lesions are on the head and neck with another 15% on shoulders, back or chest, BCCs and SCCs of the skin can have a significant impact on the appearance and quality of life of the afflicted patient.

In keratinocytes the calcium influx signal is profoundly anti-proliferative and pro-differentiating, thus enhancing this signal should be a valuable means to treat epidermal hyperplasia in psoriasis, BCC, and SCC. However, the molecular basis for this signal, the plasma membrane calcium “gatekeeper”, is unknown. One possibility identity for the keratinocyte calcium gatekeeper has been suggested by the recent cloning from various calcium transporting epithelia of two channels belonging to the OTRPC channel family, hECaC1 and hECaC2 (epithelial calcium channel 1 and 2; Hoenderop et al, 1999 J Biol Chem 274:8375-8378; Peng et al, 1999 J Biol Chem 274:22739-22746; Peng et al, 2000 J Biol Chem 275:28186-28194). The OTRPC cationic channel family includes the vanilloid receptors (VR1, VRL-1), transient receptor potential (TRP) channels, and the osmolarity-sensitive channel OTRPC4 (Harteneck et al, 2000 Trends Neurosci 23:159-166).

The genes for hECaC1 and hECaC2 are juxtaposed on chromosome 7q35 (Hoenderop et al, 2001 J Physiol 537:747-761), and their expression pattern is consistent with them having important roles in systemic calcium homeostasis. In mammals ECaC1 appears to be the principal route for apical calcium reabsorption in the kidney cortex, and it is expressed in other major sites for calcium absorption including duodenum, jejunum, and colon. ECaC1 expression has also been reported for placenta, pancreas, testis, prostate and brain (Hoenderop et al, 2001 J Physiol 537:747-761; Müller et al, 2000 Genomics 67:48-53; Peng et al, 2000 Biochem Biophys Res Commun 278:326-332). Mammalian ECaC2 expression follows a pattern similar to that of ECaC1 with expression in stomach also being reported (Hoenderop et al, 2001 J Physiol 537:747-761; Peng et al, 2001 Genomics 76:99-109; Barley et al, 2001 Am J Physiol 280:G285-G290).

The expression of ECaC channels in epithelia, and particularly their expression in Vitamin D (VitD) responsive tissues such as the kidney and proximal small intestine, suggest they might be present in human epidermal keratinocytes where they could act as an important calcium influx mechanism. Also, although skin does not contribute significantly to systemic calcium homeostasis, it is clearly sensitive to both extracellular calcium and VitD as anti-proliferative/pro-differentiating (Bollinger-Bollag and Bollag, 2001 Mol Cell Endo 177:173-182). Thus we have looked for the expression of either hECaC1 or hECaC2 in cultured primary human epidermal keratinocytes and skin, and we have assessed if one or both hECaCs in keratinocytes and skin might be increased as a function of cell differentiation in response to VitD or elevated extracellular calcium. Our initial results, reported herein, indicate that hECaC2 is expressed preferentially over hECaC1 in human keratinocytes and skin, that keratinocyte differentiation is accompanied by increased expression of hECaC2, and that hECaC2 expression in both cultured keratinocytes and skin is particularly responsive to VitD. Thus hECaC2 is a good candidate for the calcium gatekeeper mechanism subserving keratinocyte differentiation in intact human skin.

### BRIEF SUMMARY OF THE INVENTION

The invention identifies a biological target, the human epithelial calcium channel type 2 (hECaC2), to control hyperplastic epidermal skin growth typified by rapidly proliferating keratinocytes. Activation or upregulation of this target constitute means for inhibiting cellular proliferation or stimulating differentiation of cells, such as keratinocytes, thus ameliorating the epidermal hyperplasia characteristic of certain skin diseases including, but not limited to, psoriasis and basal and squamous cell carcinomas. Moreover, the inhibition or down-regulation of hECaC2 activity constitutes a means for stimulating cellular, e.g. keratinocyte, proliferation, thus inducing more efficient wound repair and skin regeneration.

### BRIEF DESCRIPTION OF THE DRAWINGS

FIG. 1 shows that application of VitD or elevated extracellular calcium to human epidermal keratinocytes causes a time-dependent increase in expression of the epithelial calcium channel hECaC2. Quantitative, TaqMan-based PCR was used to assess changes in mRNA levels for hECaC2 (normalized to GAPDH) from keratinocyte cultures grown in 2 mM calcium or 1 μM VitD over 1 and 3 days. mRNA levels are expressed as fold-changes versus levels measured in sibling cultures grown for 1 and 3 days in basal calcium (0.06 mM) and no VitD. VitD caused greater increases in hECaC2 expression than did calcium. When applied together for 3 days calcium and VitD had a synergistic effect on hECaC2 levels. Note differences in scaling of y-axes. All increases are significant from control with the exception of calcium at 1 day. Results are mean-±SEM from three experiments.

FIG. 2 shows expression levels for hECaC2 are highly consistent in skin biopsies from among healthy donors. Skin biopsies were taken from healthy donors (via Cooperative Human Tissue Network, Columbus, Ohio) and immediately placed in RNALater to preserve mRNA integrity. Quantitative PCR was then used to assess variability in expression levels of hECaC2 in these biopsies. Results are shown as high-low box-plots with individual values designated by symbols in each of the plots. hECaC2 expression for single biopsies from 4 separate donors are shown.

FIG. 3 indicates that there is a decrease in hECaC2 expression levels in normal skin biopsies grown for 3 days in explant culture. Skin biopsies were taken from healthy donors and immediately placed in RNALater to preserve mRNA integrity (day−1), or they were shipped overnight in EpiLife medium and then placed in RNALater (day 0), or were grown in culture in EpiLife medium with 1.4 mM calcium for the time indicated (1 or 3 days) before mRNA preservation. Results are mean±SEM for three experiments.

FIG. 4 shows that VitD application to cultured human skin biopsies increases expression of hECaC2 in a time-dependent manner. Skin biopsies were taken from healthy donors and cultured for the indicated times with 1 μM VitD or with vehicle added to the culture medium. mRNA levels for hECaC2 were measured by quantitative PCR, normalized to GAPDH, and fold changes in expression were calculated relative to vehicle treated controls. Results are mean±SEM from two experiments.

FIG. 5 shows a model for manipulating hECaC2 activity and/or expression in order to control growth of human epidermal keratinocytes. FIG. 5A shows the basic model of how hECaC2 promotes keratinocyte differentiation while inhibiting proliferation. FIG. 5B shows that compounds that stimulate hECaC2 activity enhance differentiation. FIG. 5C shows that over-expression of hECaC2 promotes calcium-induced keratinocyte differentiation.

### DETAILED DESCRIPTION OF THE INVENTION

The inventors have discovered that human keratinocytes and skin preferentially express hECaC2, human epithelial calcium channel type 2, rather than the closely related hECaC1. Further they show that hECaC2 expression is upregulated both during calcium or VitD induced differentiation of cultured human keratinocytes, and when VitD is applied to human skin biopsies growing in explant culture. hECaC1 is not expressed in keratinocytes or skin under any of these conditions. Thus the evidence the inventors present indicates that hECaC2 is the calcium “gatekeeper” responsible for calcium entry into keratinocytes. This calcium entry then modulates keratinocyte differentiation and proliferation, and therefore the growth of the skin epidermis. The inventors have molecules that control the growth of cells, such as keratinocytes, and thus provide very attractive targets for therapeutic drug development.

The term “differentiation” includes the sum of the developmental processes whereby apparently unspecialized cells, tissues, and structures attain their adult form and function. For keratinocytes in the skin these processes include: reduction in rate of cell division and eventual cessation of division; a change in shape from columnar to polygonal; increased synthesis of certain keratin protein subtypes (e.g., keratins 1 and 10) which aggregate to form keratin filaments; increased expression of certain enzymes and structural proteins such as transglutaminase-1 (TG-1) and involucrin (INV); and, eventually the enzyme-induced degradation of nuclei and organelles. Differentiation of keratinocytes in culture includes slowing and eventual cessation of cell division, shape change from round to more elliptical and flattened, increased synthesis of differentiation-specific keratins, and increased expression of INV and TG-1.

The term “proliferation” includes the rapid and repeated production of new cells by a succession of cell divisions. For keratinocytes in skin and culture this includes preferential synthesis of certain keratin protein subtypes (e.g., keratins 5 and 16).

Diseases characterized by pathological cellular proliferation occurring in tissues expressing hECaC2 include hyperplasias and cancers of the bladder, breast, colon, lung, ovaries, pancreas, prostate, small intestine, stomach, thymus and uterus.

Diseases of the epidermis characterized by pathologic hyperplasia include psoriasis and other psoriaform dermatoses, atopic dermatitis, epidermal nevus, acanthomas, epidermal dysplasias, intraepidermal carcinomas, and basal or squamous cell carcinomas (Skin Pathology 1998 Weedon (ed) Harcourt Brace).

Diseases characterized by a lack of cellular differentiation occurring in tissues expressing hECaC2 include hyperplasias and cancers of the bladder, breast, colon, lung, ovaries, pancreas, prostate, small intestine, stomach, thymus and uterus.

Inhibition of cellular differentiation may be desirable in the following conditions: wound repair and tissue regeneration.

The term “hECaC2 polypeptide” refers to the native hECaC2 polypeptide (also known as hCaT1), such as that described by Peng et al, 2000 Biochem Biophys Res Commun 278:326-332, and also to structurally similar polypeptides that have an hECaC2 activity. For instance, it encompasses hECaC2 polypeptides having enhanced hECaC2 activity, or those than have decreased hECaC2 activity. The nucleic acid sequence of hECaC2 has been published and is available as accession number AF365927. The database for the above accession number is National Center for Biotechnology Information (NCBI), which may be accessed via the www at ncbi.nlm.nih.gov/entrez/query.fcgi?db=PubMed.

The term “antibody” encompasses immunoglobulins, including natural Igs such as IgA, IgE, IgG and IgM, and also engineered antibodies such as chimeric antibodies or multi—or bispecific antibodies, as well as functional fragments of antibodies, such as Fab.

The term “T-cell” is well known in the art, and includes T helper cells as well as cytotoxic T cells.

Methods for configuring a coding sequence, such as a sequence encoding an hECaC2 polypeptide, and regulatory sequences, such as promoters, ribosome binding sites or other regulatory sequences are well known in the art, as are methods of introducing such sequences into vectors or host cells. Inducible promoters are also known in the art. Reference is also made to Current Protocols in Molecular Biology (1987-2002), vols. 1-4, especially chapters 9 and 16.

Antisense oligonucleotides or antisense nucleic acids are well known in the art, as are methods of selecting, modifying and administering such antisense oligonucleotides. Design of antisense oligonucleotides and methods for their administration also described by U.S. Pat No. 6,159,946 and 6,365,345 which are hereby incorporated by reference.

Post-transcriptional gene silencing via RNA interference (RNAi) is accomplished by introduction of small (approximately 21 base pair) interfering RNA (siRNA) duplexes into mammalian cells. The siRNA duplex comprises a sequence (and its complement) identical to a unique portion of the gene targeted for silencing, as well as symmetrical 2-nucleotide 3′ overhangs. Upon entering the target cell siRNA incorporates into the RNA-induced silencing complex. Via base-pair interactions the siRNA guides the complex to the homologous endogenous mRNA transcript, which the complex then cleaves approximately 12 nucleotides from the 3′ terminus of the siRNA. This cleavage prevents translation of the endogenous target mRNA.

siRNAs for hECaC2 can be designed as described (Elbashir SM, et al., 2001 Nature 411: 494-498) and a BLAST search against the human genome database can then be performed to eliminate those sequences which have significant homology to other genes. The following duplexes are candidate siRNAs for hECaC2, and they represent sense and antisense sequence segments of the target mRNA:

```
        CCAGAACATGAACCTGGTGdTdT
dTdTGGUCUUGUACUUGGACCAC
        GCTACTTCAGGAAGCCTACdTdT
dTdTCGAUGAAGUCCUUCGGAUG
        GACAGGCAAGATCTCAACCdTdT
dTdTCUGUCCGUUCUAGAGUUGG
```

siRNA is introduced into cells by standard mammalian cell transfection protocols and RNAi is then confirmed by RT-PCR or Northern analysis of total RNA, or by Western or immunofluorescent analysis for the target protein.

Transgenic or animals with knock out mutations are well known in the art. Reference is also made to Current Protocols in Molecular Biology (1987-2002), vols 1-4, especially vol. 4, chapter 23.

The nucleic acid sequence of native hECaC2 may be modified and sequences encoding hECaC2 polypeptides having functional activity selected. Methods of mutagenizing a cloned nucleic acid sequences are well known and are also described by Current Protocols in Molecular Biology (1987-2002), vols. 1-4, especially vol. 1, chapter 8. Structurally similar hECaC2 nucleic acid sequences encoding functional polypeptides may be characterized by their ability to hybridize under stringent conditions to the native hECaC2 nucleic acid sequence. Such hybridization conditions may comprise hybridization at 5×SSC at a temperature of about 50 to 68° C. can be employed for the hybridization reaction. Washing may be performed using 2×SSC and optionally followed by washing using 0.5×SSC. For even higher stringency, the hybridization temperature may be raised to 68° C. or washing may be performed in a salt solution of 0.1×SSC. Other conventional hybridization procedures and conditions may also be used as described by Current Protocols in Molecular Biology, (1987-2002), see e.g. Chapter 2. Alternatively, variant nucleic acid sequences encoding hECaC2 polypeptides may be characterized by a particular degree of sequence similarity for instance, at least 60%, 70%, 80%, 90%, 95% or 99% similarity to the nucleic acid sequence of hECaC2. Such similarity may be determined by an algorithm, such as those described by Current Protocols in Molecular Biology, vol. 4, chapter 19 (1987-2002). Homology, sequence similarity or sequence identity of nucleotide or amino acid sequences may also be determined conventionally by using known software or computer programs such as the BestFit or Gap pairwise comparison programs (GCG Wisconsin Package, Genetics Computer Group, 575 Science Drive, Madison, Wis. 53711). BestFit uses the local homology algorithm of Smith and Waterman, Advances in Applied Mathematics 2: 482-489 (1981), to find the best segment of identity or similarity between two sequences. Gap performs global alignments: all of one sequence with all of another similar sequence using the method of Needleman and Wunsch, J. Mol. Biol. 48:443-453 (1970). When using a sequence alignment program such as BestFit, to determine the degree of sequence homology, similarity or identity, the default setting may be used, or an appropriate scoring matrix may be selected to optimize identity, similarity or homology scores. Similarly, when using a program such as BestFit to determine sequence identity, similarity or homology between two different amino acid sequences, the default settings may be used, or an appropriate scoring matrix, such as blosum45 or blosum80, may be selected to optimize identity, similarity or homology scores.

Functional and structural hECaC2 variants may be selected by inserting a nucleic acid encoding an hECaC2 polypeptide into a suitable host cell and measuring hECaC2 ion channel response. Methods for measuring hECaC2 ion channel response or hECaC2 functional activity are known in the art. Reference is also made to Patch-Clamp Applications and Protocols (1995) Boulton, Baker, Walz (eds) Humana Press Single-channel recording 1995 Sakmann and Neher (eds) Plenum Press.

Drugs and agents for partially controlling hyperplasia, cellular proliferation, or for modulating T cells are known in the art.

A pharmaceutical composition is a composition suitable for administration to a subject and may encompass suitable buffers, excipients, or carriers. Such a composition is generally aseptic or sterile and may be compounded for administration in a manner known in the art, for instance, by formulation as a parenteral, oral, intraocular, subcutaneous, inhaled, intramuscular, intravenous, transdermal, topical form or in a form suitable for application to a mucous membrane. Such a composition may, optionally, contain other active ingredients, such as drugs or agents that facilitate (or inhibit) cellular proliferation and/or differentiation. For instance, it may contain an immunosuppressant such as cyclosporin. It may also contain suitable palliative ingredients, such as an analgesic. It may also be in the form of a salve, ointment or lotion. Such compositions may be in the form of a cosmetic or in a form for dermal application, such as a cream or lotion.

The inventors have discovered that hECaC2 is expressed in dermal cells, such as human epidermal keratinocytes, as a function of the growth state of these cells, and also that this expression is increased by both VitD and elevated extracellular calcium. Based on these observations and data, the invention provides a means for inhibiting hyperplastic epidermal keratinocyte growth typical of diseases including psoriasis, and basal or squamous cell carcinomas. The invention thus provides a means for ameliorating symptoms and progression of these diseases.

Inhibition of keratinocyte proliferation and stimulation of differentiation can be accomplished by stimulating epidermal keratinocyte hECaC2 activity or upregulating its level of expression. hECaC2 activity can be stimulated by topical or systemic application of pharmacological agents which increase any of the following hECaC2 single channel parameters: single channel conductance, mean open time, opening frequency, or sensitivity to calcium-induced activation. hECaC2 activity can be stimulated by topical or systemic application of pharmacological agents which decrease any of the following hECaC2 single channel parameters: mean closed time.

Up-regulating hECaC2 expression in epidermal keratinocytes in vivo can be accomplished by introduction into these cells of self-promoting cDNA for the full-length hECaC2 gene. hECaC2 up-regulation can also be accomplished by introduction into these cells of cDNA for the full-length hECaC2 gene under the control of an inducible promoter, followed by induction of this promoter's activity. As shown herein hECaC2 expression in human keratinocytes can be stimulated by elevated extracellular calcium concentrations and by VitD. Up-regulation of hECaC2 expression can also be accomplished by stimulating the endogenous signaling pathways and promoter(s) which activate hECaC2 expression in human epidermal keratinocytes. This upregulation could involve application of peptide growth factors to stimulate hECaC2 expression via intracellular signaling pathways.

The inventors have also identified a means to stimulate keratinocyte proliferation to effect wound repair or skin regeneration, that is by inhibiting hECaC2 activity or downregulating its level of expression. hECaC2 activity can be inhibited by topical or systemic application of pharmacological agents which decrease any of the following hECaC2 single channel parameters: single channel conductance, mean open time, opening frequency, or sensitivity to calcium-induced activation. hECaC2 activity can be inhibited by topical or systemic application of pharmacological agents which increase any of the following hECaC2 single channel parameters: mean closed time.

Down-regulating hECaC2 expression in epidermal keratinocytes in vivo can be accomplished by introduction into these cells of antisense oligonucleotides for the hECaC2 gene. Downregulation of hECaC2 expression can also be accomplished by inhibiting the endogenous signaling pathways and promoter(s) which activate hECaC2 expression in human epidermal keratinocytes. This downregulation could involve pharmacologic inactivation of the intracellular signaling molecules known to transduce growth factor induced upregulation of hECaC2 in vivo.

Methods for screening ligands which activate or inhibit hECaC2 activity include, contacting a cell expressing hECaC2 with a compound and measuring ion channel response, levels of mRNA encoding hECaC2 or levels of hECaC2 expression.

Methods for screening functionally active hECaC2 polypeptides include, expressing a nucleic acid encoding an hECaC2 polypeptide in a cell, contacting said cell with a drug or agent known to activate hECaC2 ion channel response and measuring hECaC2 activity. Transgenic animals expressing hECaC2 or variants of hECaC2 may be employed to measure or investigate hECaC2 activity. For instance, a transgenic animal expressing a variant of an hECaC2 with lowered functional activity may be used to evaluate the effects of pharmaceuticals on skin disorders. Transgenic animals with highly active forms of hECaC2 polypeptide may be used to evaluate the effects of anti-proliferative drugs or agents.

### EXAMPLES

### Example 1

hECaC2, but not hECaC1, is Expressed in Human Keratinocytes and This Expression is Increased by Both Vitamin D and Elevated Calcium, Two Agents Which Differentiate Human Keratinocytes.

There is currently no published data on expression of either hECaC1 or hECaC2 in either primary human keratinocytes or human skin, although murine ECaC2 expression has been reported for mouse skin (Weber et al, 2001 Biochem Biophys Res Commun 289:1287-1294). Therefore, the inventors investigated if keratinocytes expressed either or both ECaC channels, and whether expression of these channels was correlated with cell differentiation stimulated by either calcium or VitD. Quantitative PCR failed to detect hECaC1 expression in keratinocyte cultures grown for 1 to 3 days under basal conditions (0.06 mM calcium, no added VitD). However, hECaC2 expression was readily detected in these same cultures. At 1 and 3 days of culture hECaC2 expression levels were 0.014±0.002% and 0.005±0.0005% of GAPDH (N=3). To check that the apparent absence of hECaC1 expression was not due to inefficient or faulty PCR primers or probe, a human kidney expression library was used as a positive control (ECaC1 expression in human kidney has been conclusively demonstrated, see references herein). hECaC 1 expression was readily detected from this library at a level of approximately 1.0% of GAPDH. Therefore, the observed absence of hECaC1 expression in keratinocytes was indeed due to the mRNA for this gene being at levels so low that they were undetectable.

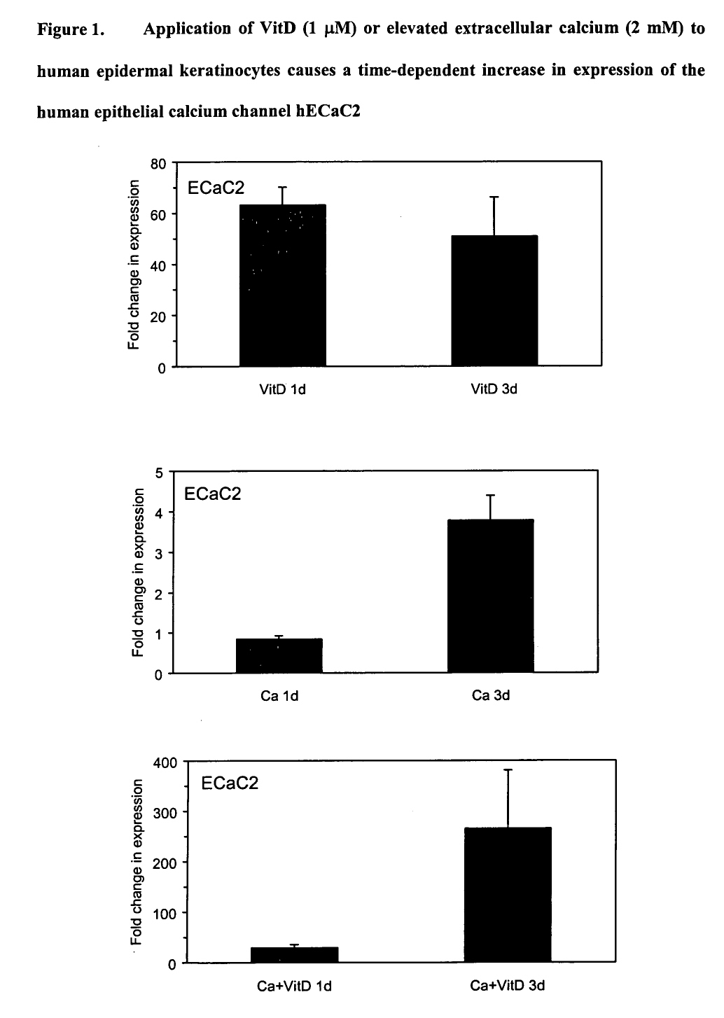

The effects of elevated calcium or VitD on expression of hECaC1 or 2 in cultured keratinocytes were investigated. Calcium and/or VitD induced changes in gene expression were expressed as fold-changes relative to untreated, time-matched cultures. hECaC1 expression was not detected at up to 3 days treatment with either 2 mM calcium or 1 μM VitD, however both agents significantly elevated hECaC2 expression (FIG. 1). One and three day treatment of keratinocytes with VitD significantly increased hECaC2 expression by 63-fold and 51-fold respectively. After 1 day in 2 mM calcium there was a small but consistent decrease in hECaC2 expression, which averaged approximately 85% of the untreated control. However, after 3 days in 2 mM calcium, hECaC2 expression significantly increased by approximately 3.8-fold.

The combination of 2 mM calcium and 1 μM VitD had a complicated effect on hECaC2 expression (FIG. 1). After 1 day exposure to calcium and VitD, hECaC2 expression was significantly increased relative to untreated cultures by 29.7-fold, an increase which was only about one-half of that observed in response to VitD alone (60-fold). At 3 days exposure, however, calcium and VitD had synergistic effects on hECaC2 expression. hECaC2 expression was significantly elevated by 266-fold relative to untreated cultures, more than 5 times the increase due to VitD alone at 3 days (51-fold), and more than would be predicted from multiplying the individual increases due to VitD and calcium.

### Example 2

Expression Levels for hECaC2 are Highly Consistent in Skin Biopsies From Among Healthy Donors.

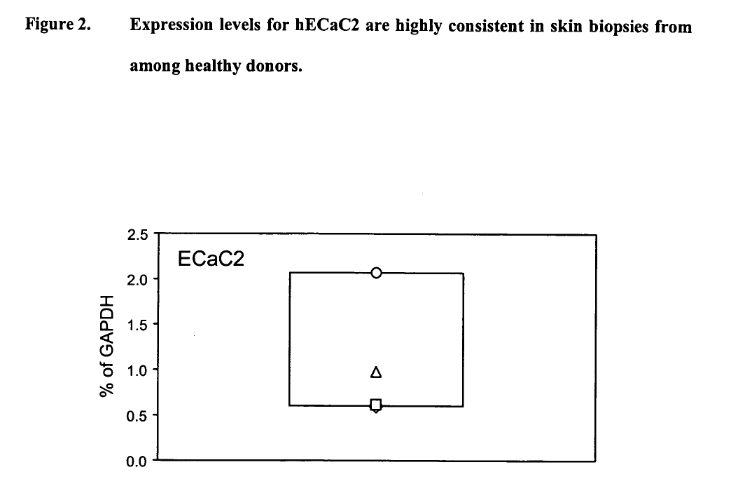

Based on the presence of hECaC2 RNA in cultured keratinocytes and the increase in this expression when keratinocytes were stimulated to differentiate, the inventors looked to see if this channel was expressed in human skin punch biopsies. Biopsies (5 mm) were taken and immediately placed in RNALater to preserve their RNA content and integrity. hECaC2 expression in these biopsies ranged from 0.6 to 2% of GAPDH expression (FIG. 2), values somewhat greater than those observed in cultured keratinocytes growing in basal calcium with no added VitD. However, keratinocytes treated with VitD alone or calcium and VitD express hECaC2 at 0.3 to 1.5% of GAPDH, values within the range for skin hECaC2 levels.

### Example 3

ECaC2 Expression Levels Decrease in Normal Skin Biopsies Grown for 3 Days in Explant Culture.

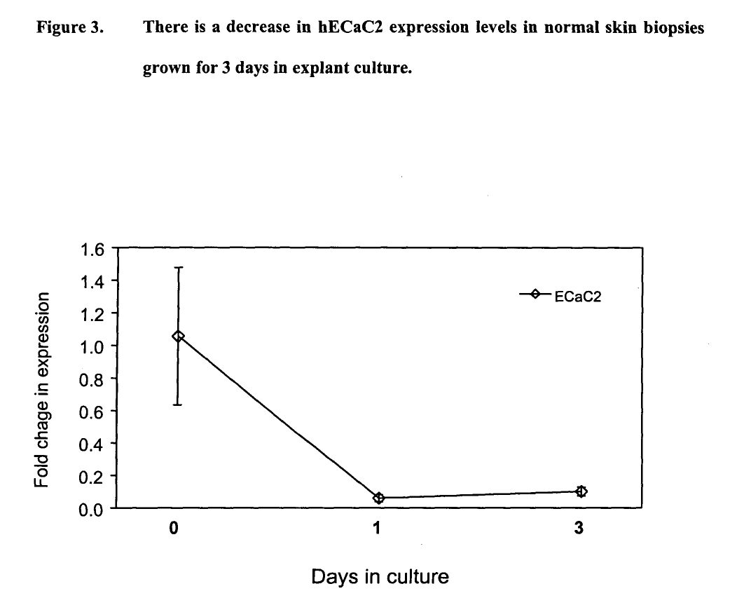

The effect of VitD on hECaC2 expression in skin explants over time in culture was next examined, but the dynamics of gene expression in this system had to first be defined. All experiments with explant, cultured skin biopsies were performed in medium containing 1.4 mM calcium, a requirement for long-term viability (Varani et al, 1993 Am J Pathol 142:189-198). Biopsies placed in RNALater at time of collection were used to define expression levels of these genes at “−1” days in culture. Biopsies for which RNA was extracted immediately upon arrival in the lab (after overnight shipping from the source) were designated as “0” days in culture. Data for days 1 and 3 are from biopsies which were then cultured in the lab. All biopsies for an experiment were from the same donor. FIG. 3 shows that hECaC2 expression significantly decreased during time in culture, falling to 6% of its day −1 value after the first day in culture, and recovering slightly to 10% at day 3.

### Example 4

Vitamin D Application to Cultured Human Skin Biopsies Increases Expression of hECaC2.

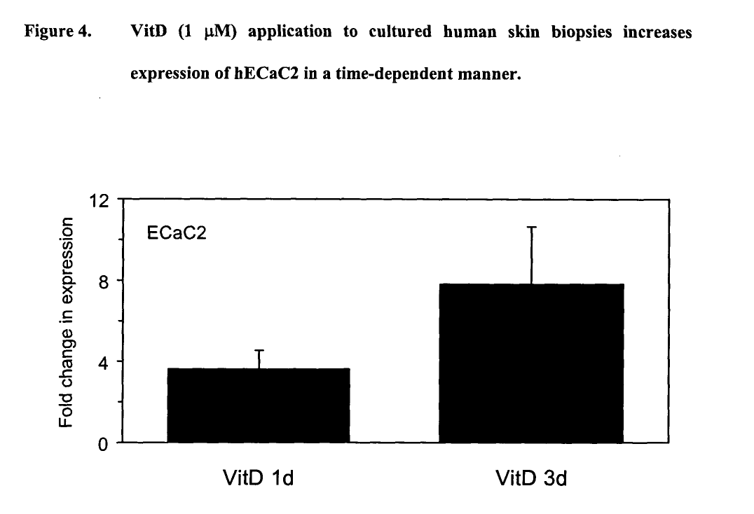

The effect of VitD on gene expression in cultured biopsies was assessed by comparing mRNA levels in the treated biopsies to levels in untreated biopsies cultured for the same number of days (FIG. 4). In response to 1 μM VitD in the culture medium hECaC2 expression increased by 3.6-fold after 1 day, and by 7.8-fold after 3 days. hECaC1 expression was not detected in any biopsy nor was expression induced in response to VitD.

### Example 5

A Model for hECaC2 Control of Keratinocyte Growth

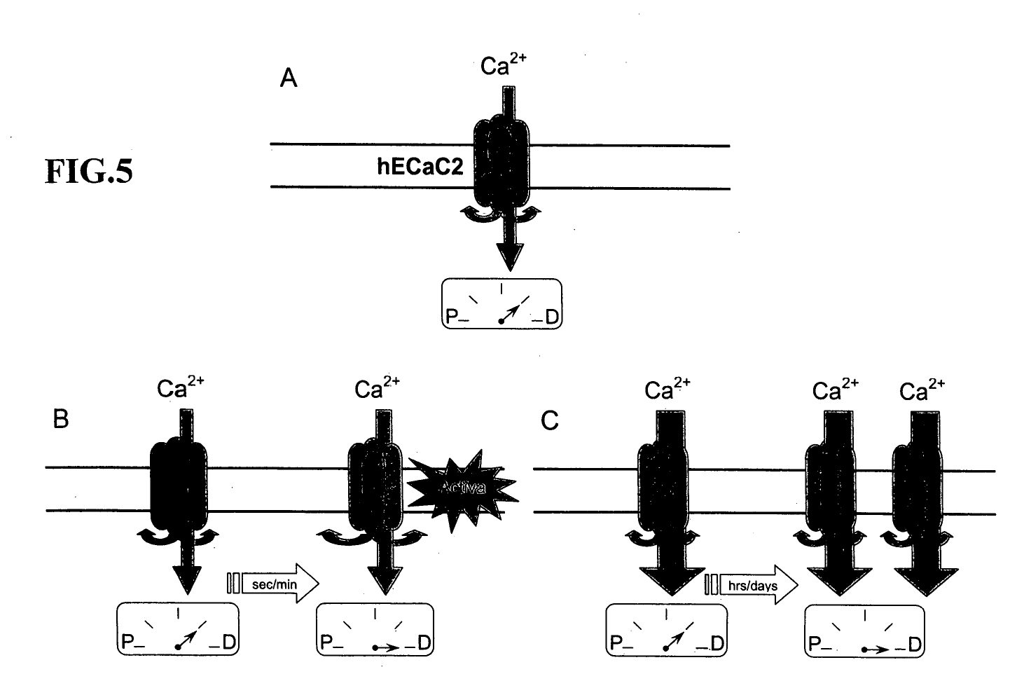

There is currently no published data on expression of either hECaC1 or hECaC2 in either primary human keratinocytes or human skin, although murine ECaC2 expression has been reported for mouse skin (Weber et al, 2001 Biochem Biophys Res Commun 289:1287-1294). Also, expression of either murine ECaC1 or 2 was shown to be unresponsive to VitD (including in skin)(Weber et al, 2001 Biochem Biophys Res Commun 289:1287-1294), although hECaC2 expression in the Caco-2 human intestinal cell line was reportedly upregulated by VitD (Wood et al, 2001 BMC Physiol 1:11-18). Results reported herein clearly show a robust and synergistic enhancement of hECaC2 expression in human keratinocytes by VitD and calcium, two important physiological regulators of keratinocyte differentiation. Further, explant skin biopsies respond to VitD with elevated hECaC2 expression within one day, and this response increases out to three days. These results, and the data here showing complete lack of hECaC1 expression in keratinocytes or skin, strongly suggests that hECaC2 is a prime candidate for the calcium gatekeeper underlying cellular differentiation in human skin (FIG. 5). Further, this study identifies control of hECaC2 expression as a potential factor in the dermatological therapeutic actions of VitD. Recently, another member of the OTRPC channel family, the thermosensitive TRPV3 channel, was shown to be expressed in human keratinocytes and skin, although its sensitivity to calcium or VitD and its role in keratinocyte differentiation are unreported (Peier et al, 2002 Science online:1073140-<zdoi;10.1126/science.1073140). Although TRPV3 and hECaC2 belong to the same channel family a BLAST alignment revealed no sequence identity at the nucleotide level. Thus hECaC2 and TRPV3 are distinct channels that may mediate very different physiologic events in skin. In conclusion, activation targeting of hECaC2 has growth inhibitory actions specific to keratinocytes and other epithelial cells, thus providing therapeutic opportunities for specifically targeting epithelial cell diseases.

The experimental strategy to validate hECaC2 as a target for controlling hyperplastic growth of human keratinocytes is based on the model shown in FIG. 5. According to this model, manipulation of hECaC2 activity and/or expression level is predicted to modulate keratinocyte growth state. For example, activators of hECaC2 should inhibit proliferation (and stimulate differentiation) as depicted in FIG. 5B, while increased hECaC2 expression would have a similar effect (FIG. 5C).

### Example 6

Drugs That Activate hECaC2 Activity Inhibit Keratinocyte Proliferation.

The model for hECaC2 function in human epidermal keratinocytes predicts that stimulation of hECaC2 activity promotes differentiation while also inhibiting proliferation. It is possible to test the effects of hECaC2 activator to see if they inhibit keratinocyte proliferation. If this is the case then they attempt to reverse this affect by co-applying compounds which block the entry of calcium through hECaC2.

### Example 7

Test to see if hECaC2 Activation Cause upregulation of Keratinocyte Differentiation Genes.

Normal keratinocyte growth consists of a balance between proliferation and differentiation (FIG. 5), with cells being biased towards differentiation as they move from the inner layers of the epidermis towards the outer stratum comeum. This bias is lost in skin diseases characterized by dysregulated epidermal growth (e.g., atopic dermatitis, psoriasis, basal cell carcinoma), in which keratinocytes both proliferate too readily and differentiate inefficiently. This data shows that increased hECaC2 expression is associated with keratinocyte differentiation, therefore it is possible that hECaC2 activity also stimulates differentiation. To test this hypothesis the inventors can use TaqMan-based quantitative RT-PCR to see if pharmacologic activation of hECaC2 results in upregulation of differentiation-specific genes in keratinocytes. These well-characterized genes include involucrin, transglutarninse-1, and peroxisome proliferator activated receptor γ.

Keratinocyte differentiation is inhibited in growth medium containing low calcium concentrations (<0.3 mM). Therefore the ability of hECaC2 activation to produce differentiation may be limited for cells cultured in low-calcium medium. For this reason the inventors also test to see if hECaC2 activation potentiates the differentiation observed when cells are grown in high-calcium (2 mM) medium. A positive result still indicates the importance of hECaC2 to controlling the balance between proliferation and differentiation.

### Example 8

Test Whether Enhancing hECaC2 Expression Inhibits Proliferation and/or Promotes Differentiation.

Enhancing hECaC2 expression, like activating native hECaC2 channels, should inhibit proliferation and possibly stimulate differentiation. To test this idea the coding region of hECaC2 is subdloned into a zinc-inducible mammalian expression vector, pMEP4, and this vector is transfected into keratinocyte and stably expressing cells are selected with gentamycin. These cells are exposed to 10-100 μM zinc-chloride for 24 hours to induce high levels of hECaC2 expression. This induced hECaC2 expression inhibits proliferation and stimulate differentiation, and it is possible to be tested with proliferation and differentiation gene expression assays, respectively.

### Example 9

Test Whether Inhibiting hECaC2 Expression Blocks Differentiation.

To inhibit hECaC2 expression cDNA encoding anti-sense nucleotides for hECaC2 is introduced into human keratinocytes. This technique is used to downregulate hECaC2 expression in keratinocytes, and these cells are also tested to see if their ability to differentiate in response to high-calcium growth medium is suppressed.

Modifications and Other Embodiments

Various modifications and variations of the invention as well as the concept of the invention are apparent to those skilled in the art without departing from the scope and spirit of the invention. Although the invention has been described in connection with specific preferred embodiments, it should be understood that the invention as claimed is not intended to be limited to such specific embodiments. Various modifications of the described modes for carrying out the invention which are obvious to those skilled in the biological, biochemical, medical, pharmaceutical, molecular biological and immunological arts and related fields are intended to be within the scope of the invention.

Incorporation by Reference

Each document, patent application or patent publication cited by or referred to in this disclosure is incorporated by reference in its entirety. Any patent document to which this application claims priority is also incorporated by reference in its entirety. Specifically, the contents of U.S. Provisional Application No. 60/404,153 are hereby incorporated by reference. Inter alia, the Applicants specifically incorporate by reference the subject matter disclosed in Embodiments 6-10, 17, 18, 22-26, 28-30,37-40, 44, 48-61 and 63-79 appearing on pages 21-26 of the above-mentioned provisional application.

1. A method for promoting differentiation of a cell or tissue comprising administering an agent that modulates the activity or expression of the human epithelial calcium channel 2 (hECaC2).

2. The method of claim 1 that stimulates hECaC2 activity or up-regulate its level of expression.

3. The method of claim 1 that comprises promoting the differentiation of a keratinocyte.

4. The method of claim 1, wherein said cell or tissue is a tumor or cancer cell or tissue.

5. The method of claim 1 that comprises treating a wound, burn, scar or keloid.

6. A method for decreasing proliferation of a cell comprising administering an agent or drug that modulates the activity or expression of the human epithelial calcium channel 2 (hECaC2).

7. The method of claim 6, wherein said cell or tissue is a hyperplastic cell or tissue.

8. The method of claim 6 that comprises decreasing the proliferation of a keratinocyte.

9. The method of claim 6, wherein said cell or tissue is a tumor or cancer cell or tissue.

10. The method of claim 6, wherein cell is a cancer cell from basal cell, breast, colon, or prostate cancer.

11. The method of claim 6 that comprises treating a wound, burn, scar or keloid.

12. A method for treating a disease characterized by hyperplastic epidermal keratinocyte growth comprising administering an agent or drug that modulates the activity of the human epithelial calcium channel 2 (hECaC2).

13. The method of claim 12, wherein said disease is psoriasis, or basal or squamous cell carcinoma.

14. A method for increasing proliferation of a cell or tissue comprising administering an agent that modulates the activity or expression of the human epithelial calcium channel 2 (hECaC2).

15. A method for inducing regeneration comprising administering an agent or drug that modulates the activity of the human epithelial calcium channel 2 (hECaC2).

16. A method for inhibiting the differentiation of a cell or tissue comprising administering an agent that modulates the activity or expression of the human epithelial calcium channel 2 (hECaC2).

17. The method of claim 16, wherein said cell or tissue is in need of repair, regeneration or growth.

18. The method of claim 16 that comprises increasing the proliferation of a skin cell.

19. The method of claim 16 that comprises increasing the proliferation of a keratinocyte.

20. The method of claim 16 that comprises treating a wound or burn.

21. The method of claim 16 comprising introducing into said cell or tissue an antisense hECaC2 nucleic acid.

22. A method to up-regulate hECaC2 expression in epithelial cells in vivo by introducing a nucleic acid encoding an hECaC2 polypeptide.

23. The method of claim 22 that comprises introducing cDNA.

24. The method of claim 22 that comprises introducing a vector comprising cDNA encoding an hECaC2 polypeptide and a promoter.

25. A method to up-regulate hECaC2 expression in epithelial cells in vivo by stimulation or induction of endogenous signaling pathways or promoter elements which control hECaC2 expression.

26. A method to inhibit hECaC2 activity or down-regulate its level of expression by introducing into a cell, which expresses an hECaC2 polypeptide, an antisense oligonucleotide, a small-interfering RNA (siRNA), or inducing the expression of an antisense nucleic acid within a cell which expresses an hECaC2 polypeptide.

27. A method to inhibit hECaC2 activity or down-regulate its level of expression by inhibiting an endogenous signaling pathway or inhibiting expression of an hECaC2 polypeptide in a cell expressing an hECaC2 polypeptide.

28. A composition comprising a drug or agent that inhibits or promotes the activity of an hECaC2 polypeptide.
